(19) United States
(12) Patent Application Publication
(10) Pub. No.: US 2002/0115922 A1
(43) Pub. Date: Aug. 22, 2002

(54) Infrared assisted monitoring of a catheter

(76) Inventors: Milton Waner, Little Rock, AR (US); Scott L. Ferguson, Vilonia, AR (US); Louis M. Fink, Little Rock, AR (US); Vladimir P. Zharov, Moscow (RU)

Correspondence Address: Ray F. Cox, Jr., Wright, Lindsey & Jennings LLP, Suite 2200, 200 West Capitol Avenue, Little Rock, AR, 72201, US

(21) Appl. No.: 09/781,391

(22) Filed: Feb. 12, 2001

(51) Int. Cl. A61B 5/05; A61B 6/00

(52) U.S. Cl. 600/407; 600/435

(57) ABSTRACT

An apparatus for the placement and monitoring of the position of an intraluminal indwelling catheter using an infrared (IR) signal encoded in the catheter and the detection of the IR signal by an IR optical detector. The IR signal may be encoded into the catheter by IR emitted from the catheter or IR reflected from the catheter. In the first category, the catheter is illuminated by IR radiation emitted from the distal end of the catheter, either by fiber optics or by a micro-diode. In the second category, the catheter is marked with regions of varying optical properties to form a pattern that is easily visualized and distinctive from nearby anatomical structures. One embodiment has a helical pattern in either one or more solid bands or a series of helically arranged dots. Other embodiments employ a pair of criss-crossing helical bands or zebra stripes. In addition to IR radiation, other electromagnetic radiation, including visible light, may be used. An alternative embodiment for an IV catheter includes a partially opaque flash chamber having a backing with optical properties that contrast with that of blood to allow the detector to image the blood filling the chamber and verify a successful insertion.

### CROSS-REFERENCE TO RELATED APPLICATIONS

Not applicable.

### STATEMENT REGARDING FEDERALLY SPONSORED RESEARCH OR DEVELOPMENT

Not applicable.

### BACKGROUND OF THE INVENTION

1. Field of the Invention

The present invention relates to catheters, and in particular, to catheters encoded with an infrared signal to allow detection of the signal, and thus the location of the catheter, by an infrared optical detector.

2. Brief Description of the Related Art

A catheter is a tubular instrument for insertion into a bodily cavity (lumen) or orifice, naturally or surgically opened. Typically a catheter consists of a cannula through which a sharp hollow needle passes. The front end of the cannula closely sheathes the needle and is tapered to slide into the patient's tissue behind the needle. The needle may be removed from the cannula. The rear body portion of the cannula may receive standard IV (intravenous) tubing. An IV catheter may also include a “flash chamber” communicating with the hollow needle. Blood filling the flash chamber signals that the needle has pierced a blood vessel.

Currently, catheters are placed by feel. Placing the catheter in the correct position is a difficult task, requiring considerable skill.

U.S. Pat. No. 5,437,290 describes a solution to the problem of positioning an intraluminal device, such as a catheter. This patent also discusses a technique of fluoroscopic imaging of radiopaque markers to position catheters.

The limitations of the prior art are overcome by the present invention as described below.

### BRIEF SUMMARY OF THE INVENTION

In the present invention, the placement and monitoring of the position of an intraluminal indwelling catheter is assisted through an infrared (IR) signal encoded in the catheter and the detection of the IR signal by an IR optical detector. Such a detector is disclosed in U.S. Pat. No. 6,032,070, the disclosure of which is incorporated herein by reference. It permits the viewing of anatomical structures, such as blood vessels, by enhancing the contrast in reflected electromagnetic radiation between the targeted structure and the surrounding tissue. Enhancing contrast may be achieved by image processing, filtering, detecting polarized light, or other techniques known in the art. Other types of optical detectors may be employed in alternative embodiments of the present invention. For example, an array of photodiodes may be employed to detect the electromagnetic radiation from the catheter. By measuring the amount of radiation received at each photodiode, the location of the source of the radiation may be determined.

The catheter of the present invention is selectively encoded with an infrared signal that is captured by the detector. The IR signal may be encoded into the catheter in a number of different ways that fall into two main categories: (1) IR emitted from the catheter or (2) IR reflected from or absorbed by the catheter. Included in the category of IR emitted from the catheter is fluorescence of one spectral range excited from fluorescent material in the catheter due to impinging radiation of another spectral range.

In the first category, the catheter may be illuminated by IR radiation emitted from the distal end of the catheter, in particular from the distal end of the cannula. This is particularly helpful in precisely detecting the location of the critical distal end of the catheter. The IR may be provided by fiber optics delivering the IR signal from a remote IR source or by one or more micro-diodes located in the distal end of the cannula.

In the second category, the catheter may be marked by a distinctive recognizable pattern with regions of varying optical properties; i.e., with contrasting reflective and absorptive properties. One embodiment would have a helical pattern in either one or more solid bands or a series of helically arranged dots on the cannula. The solid bands could include, for example, “zebra stripes” or similar strongly identifiable markings. Another embodiment would employ a pair of criss-crossing helical bands. The intent is to produce a pattern that is easily visualized and distinctive from nearby anatomical structures. In order to differentiate the distal end of the catheter from the proximal end, the pattern may be more intense at the distal end and less intense as the pattern proceeds toward the proximal end. As an example, a pattern of solid bands may be more densely disposed toward the distal and less densely disposed toward the proximal end. Since a IV catheter would be used in or near blood vessels, it is important that the patterns be visible against blood. While the preferred embodiment of the invention would use IR radiation, other electromagnetic radiation, including visible light, could be effective in particular uses.

A significant use for the present invention would be the placement of an IV catheter. Other uses would include the detection of plaque or irregularities in the walls of blood vessels. Furthermore, dyes conjugated to antibodies could be detected by using the illumination of the present invention as a source for spectrophotometry. The present invention could be used both to detect and to excite such compounds to allow visualization or selective destruction. The present invention is not limited to IV catheters but may also be employed with catheter used in laser surgery in order to place the distal end of the catheter and thus an optical fiber in the proper location with respect to a tumor or other body structure receiving laser therapy.

An alternative embodiment of the present invention includes a partially opaque flash chamber. Since most flash chambers are transparent, it would be difficult to visualize the blood filling the chamber. A white, or otherwise opaque, backing to the flash chamber would allow the detector to image the blood filling the chamber and verify a successful insertion. The opaque backing may optically reflective or absorptive in the spectral range of interest so long as it contrasts with the optical properties of the blood.

It is therefore an object of the present invention to provide for an intraluminal indwelling catheter having an IR signal encoded in the catheter.

It is a further object of the present invention to provide for such a catheter wherein the IR signal is detected by an IR optical detector so as to determine the location of the catheter.

It is also an object of the present invention to provide such a catheter wherein the IR signal is encoded by IR radiation emitted from the distal end of the catheter, such as by fiber optics delivering the IR signal from a remote IR source or by a micro-diode located in the distal end of the catheter.

It is additionally an object of the present invention to provide such a catheter wherein the IR signal is encoded by IR reflected from the catheter, and in particular, wherein the catheter is marked by a distinctive recognizable pattern with regions of varying optical properties; i.e., with contrasting reflective and absorptive properties.

These and other features, objects and advantages of the present invention will become better understood from a consideration of the following detailed description of the preferred embodiments and appended claims in conjunction with the drawings as described following:

### BRIEF DESCRIPTION OF THE SEVERAL VIEWS OF THE DRAWINGS

FIG. 1 is a perspective view of the catheter of the present invention. The catheter needle is enclosed in a cannula.

FIG. 2 is an exploded perspective view of the catheter with the catheter needle withdrawn from the cannula.

FIG. 3 is a vertical cross section through the catheter of FIG. 1.

FIG. 4 is a cross section along the line 4-4 of FIG. 3.

FIG. 5 is partial exploded view of the body of the cannula showing one embodiment of the means for coupling an IR signal into the cannula.

FIG. 6 is a perspective view of one embodiment of the cannula in which an IR signal is encoded in a pair of helical bands having IR reflective or absorptive properties differing from such properties of the cannula.

FIG. 7 is a section of FIG. 6 as indicated by 7-7 on FIG. 6. FIG. 7 illustrates one embodiment in which the helical bands are continuous.

FIG. 8 is a section of FIG. 6 showing an alternative embodiment to that of FIG. 7 in which the helical bands are formed of a series of dots.

FIG. 9 is a perspective view of the catheter of the present invention illustrating the insertion of the catheter into the arm of a patient and the detection of the location of the catheter by an IR optical detector.

FIG. 10 is a perspective view of a flash chamber having a contrasting backing to allow the detector to image the blood filling the chamber and verify a successful insertion.

FIG. 11 is a perspective view of an alternative embodiment of the present invention in which an IR signal is coupled to the catheter so as to illuminate the distal end of the cannula by an IR signal from an external IR source or by a micro-diode located at the distal end of the cannula.

FIG. 12 is a partial vertical cross section through the cannula illustrating the coupling of an IR signal from an external source to the distal end of the cannula.

FIG. 13 is a vertical view of the distal end of the cannula illustrating the placement of a micro-diode at the distal end of the cannula.

FIG. 14 is a cross section of FIG. 13 along the line 14-14 illustrating the placement of a micro-diode at the distal end of a cannula.

### DETAILED DESCRIPTION OF THE INVENTION

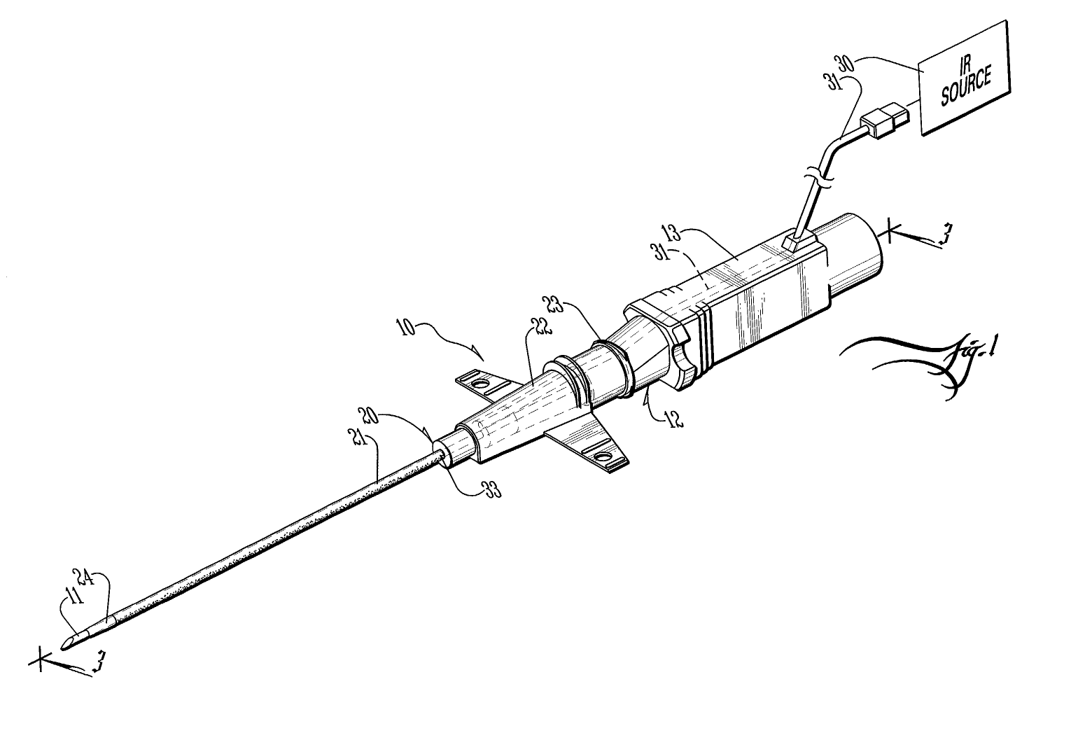

With reference to FIGS. 1 and 2, the preferred embodiment of the present invention may be described. Typically, an intraluminal, indwelling catheter 10 includes a hollow needle 11 communicating with a hollow body 12 which may in turn communicate with a hollow flash chamber 13. The catheter 10 is sheathed with a cannula 20 comprising a needle sheathing portion 21 and a body sheathing portion 22. The distal end 24 of the cannula is tapered to slide into the patient's tissue behind the sharp hollow needle 11 which protrudes from the distal end 24 of the cannula 20. The proximal end 23 of the cannula 20 may receive standard intravenous tubing (not shown) in an IV catheter.

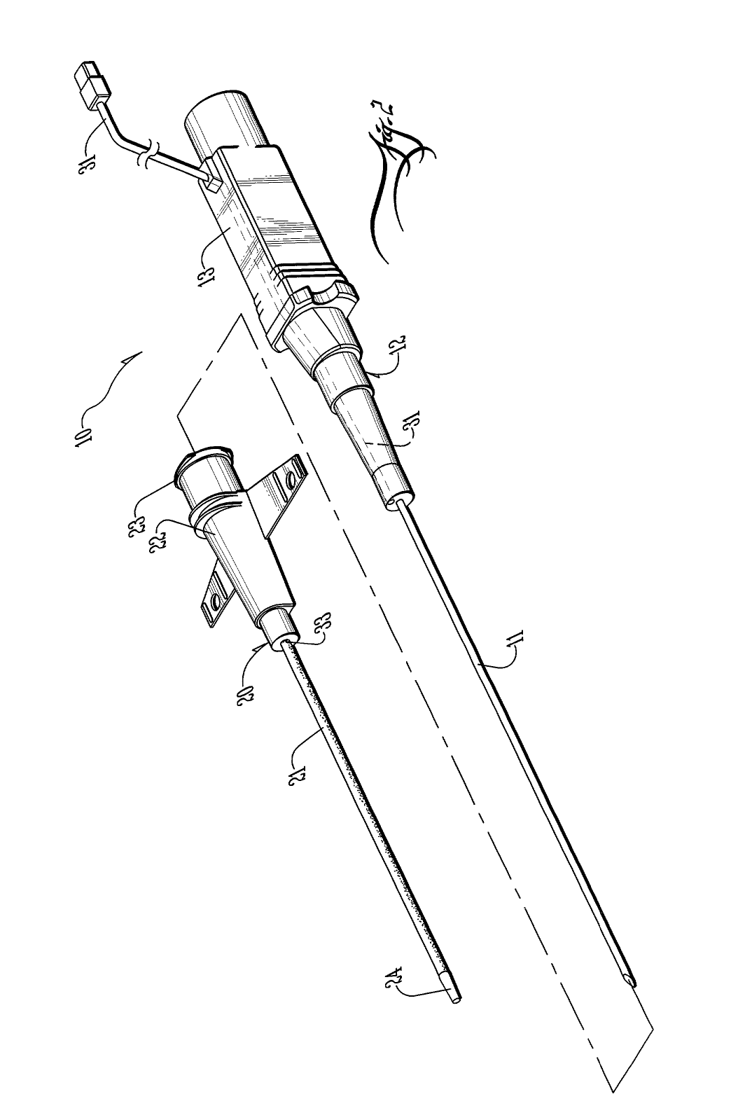

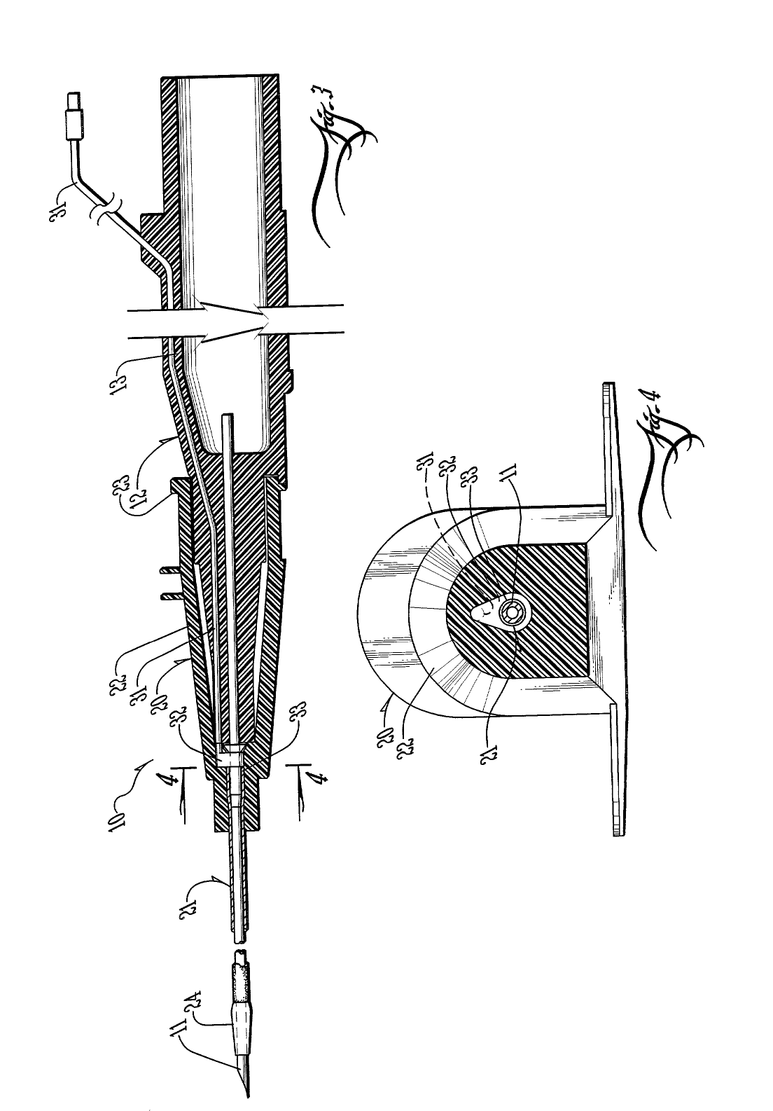

One embodiment of the invention employs infrared (JR) radiation emitted from the needle sheathing portion 21 of the cannula 20 to assist in the location of the distal end 24 of the cannula 20 so as to assist in the proper placement of the catheter 10. Various means may be employed to illuminate the cannula 20. In one embodiment illustrated with reference to FIGS. 1-5, a remote IR source 30 from, e.g., an infrared laser or light emitting diode, may be transmitted by a fiber optic cable 31 embedded in the walls of the flash chamber 13 and hollow body 12 of the catheter 10 to a coupling element 32 positioned between the hollow body 12 and the body sheathing portion 22 of the cannula 20. The coupling element 32 illuminates the proximal end 33 of the needle sheathing portion 21 of the cannula 20. The walls of the hollow needle sheathing portion 21 of the cannula 20 then act as a light guide to transmit the IR radiation to the distal end 24 where the IR escapes from the cannula 20, thus providing a source of IR emanating from the distal end 24. Various other means of coupling the IR radiation to the cannula 20 known in the art are contemplated as being within the scope of the present invention. For example, the fiber optic cable 31 may be coupled directly to the body sheathing portion 22 of the cannula 20. The IR radiation may be transmitted directly to the distal end 33 of the needle sheathing portion 21 of the cannula 20 without or without a coupling element so long as the distal end 24 is illuminated by the IR. A diffusive tip may be employed with the optical fiber. In addition, one or more optical fibers may be employed to emit radiation from a plurality of diffusive tips along the length of the cannula 20. The points from which radiation is emitted, either from a optical fiber or from one or more mini-LEDs, may be formed into various patterns, for example, one or more linear arrays along the length of the cannula 20. Furthermore, the light emitting characteristics of the sources of radiation may be adjusted so that the pattern is more intense toward the distal end 24 of the cannula 20 and less intense toward the proximal end so that the distal end 24 may be easily distinguished while the orientation of the cannula 20 is also clearly distinguishable. Modulated the radiation signal may enhance the sensitivity by which the radiation signal is detected. Detection may be, for example, by known detection techniques such as phase locked loop circuitry.

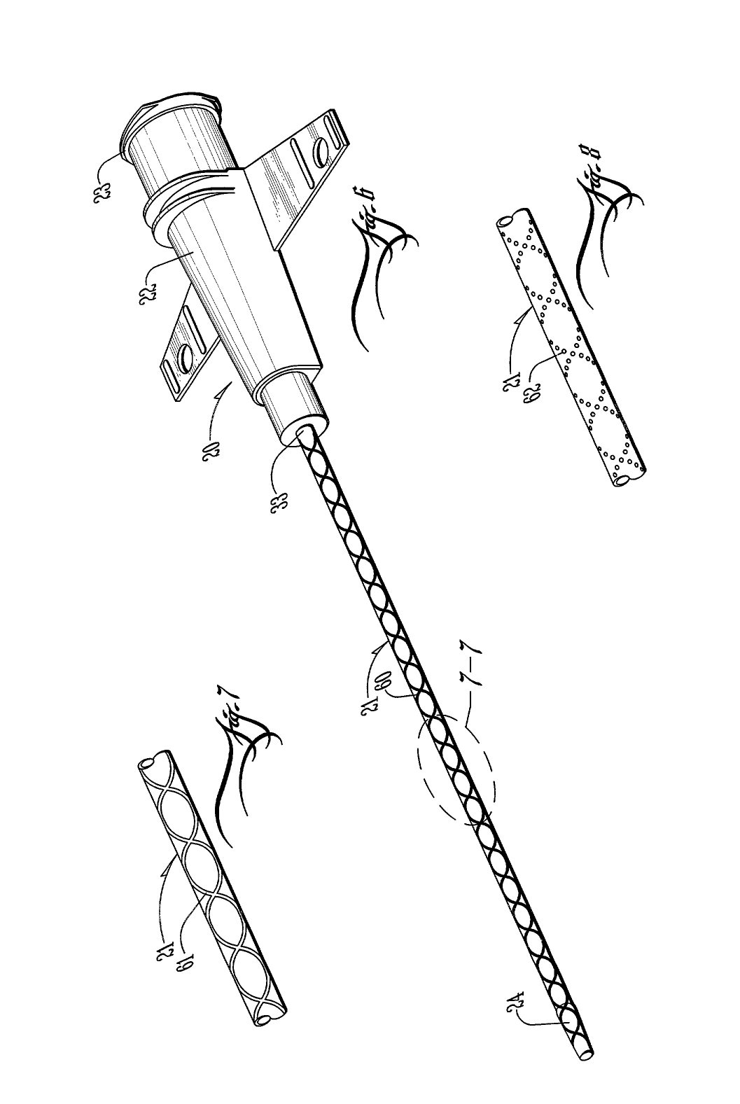

As illustrated by FIG. 9, the present invention is used to assist in the proper placement of the catheter 10. As an example, FIG. 9 shows a catheter 10 being placed in the arm 40 of a patient. The intent is to precisely place the distal end of the catheter's needle 11 within the blood vessel 41. In the embodiment of the invention described above, the distal end 24 of the cannula 20 is illuminated by IR source 30. An IR image detector 50 receives and enhances the IR radiation from the distal end 24 and process the image of the distal end 24 for viewing on the monitor 51. The physician is thus provided with guidance for the precise placement of the catheter.

A suitable IR image detector is disclosed in U.S. Pat. No. 6,032,070, although the present invention is not limited to this IR detector. In other applications, the present invention may be used with other image detecting and enhancing means, including those that operate in other portions of the electromagnetic spectrum. In such cases, the catheter may be illuminated by other electromagnetic radiation than IR.

The radiation emitted from the distal end 24 of the cannula 20 may be detected by a photodiode array, such as a ring shape. At least four photodiodes would be desirable. The radiation emitted through the tissue of the patient will be absorbed and diminished in proportion to the length of the tissue being traversed. The location of a source of radiation on the cannula 20 may then be pinpointed by the relative intensity of the irradiation at each of the photodiodes. The location information extracted from such a photodiode may be displayed or communicated to the user in a number of ways, for example, by a liquid crystal display or even by a sound of varying intensity and tone to verify the position of the distal end 24 of the cannula 20 at the desired location.

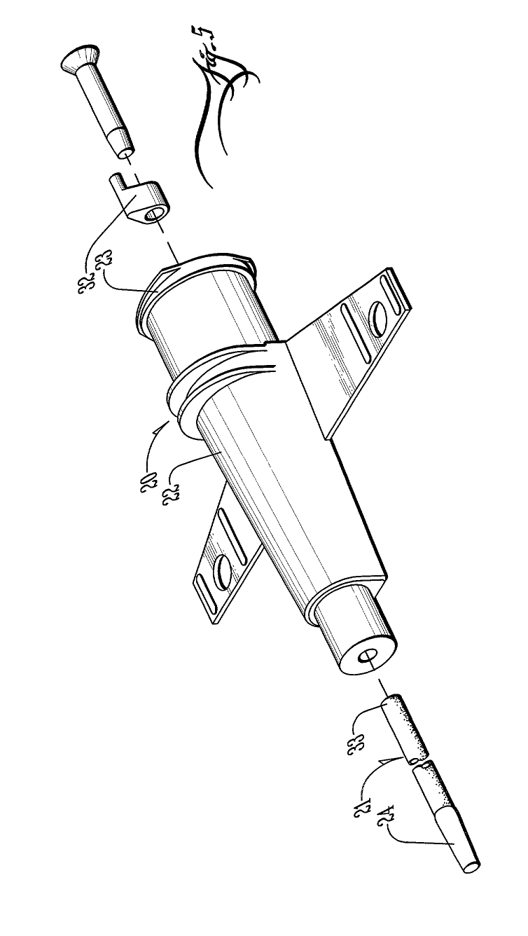

An alternative embodiment of the present invention employs IR or other electromagnetic radiation reflected from or absorbed by the catheter 10 rather than radiation emitted from the catheter 10. This alternative embodiment is discussed with reference to FIGS. 6-8. In this alternative embodiment, the catheter 10 and in particular, the needle sheathing portion 21 of the cannula 20 is marked by a distinctive recognizable pattern 60 with regions of varying optical properties; i.e., with contrasting reflective and absorptive properties. In the embodiment of FIG. 7, the pattern 61 is a helical pattern in either one or more solid bands or a pair of criss-crossing helical bands. In the embodiment of FIG. 8, the pattern 62 is a series of helically arranged dots. While the illustrated patterns are considered to be effective in the practice of the present invention, other patterns could be employed, such as “zebra stripes.” Any pattern 60 that is easily visualized and distinctive from nearby anatomical structures is contemplated as being within the scope of the present invention. Since an IV catheter would be used in or near blood vessels, it is important that the patterns in this situation be visible against blood. In certain applications, it would be desirable for the distal end 24 of the cannula 20 to be distinguished while also allowing the orientation of the cannula 20 to be visualized also. For this purpose, the pattern 60 may be more intense, for example by making the pattern more dense, near the distal end 24 and less intense, i.e., less dense, toward the proximal end.

In this alternative embodiment, the catheter 10 would be used as shown in FIG. 9 and as described above with reference to the embodiment in which the distal end 24 of the cannula 20 is illuminated by IR radiation. In the alternative embodiment, however, the IR source 30 is not coupled to the catheter 10, but instead an external IR source 70 is employed to provided IR radiation which illuminates the patient's arm 40 and thus the patterned cannula 20. The IR reflected from the patterned cannula 20 is detected and imaged by the image detector 50. In another embodiment, the cannula 20 may incorporate fluorescent materials and the external source 70 may illuminate the cannula 20 with radiation of a spectra causing the fluorescent materials to fluoresce in a spectral range that is detectable by the imaging device 50.

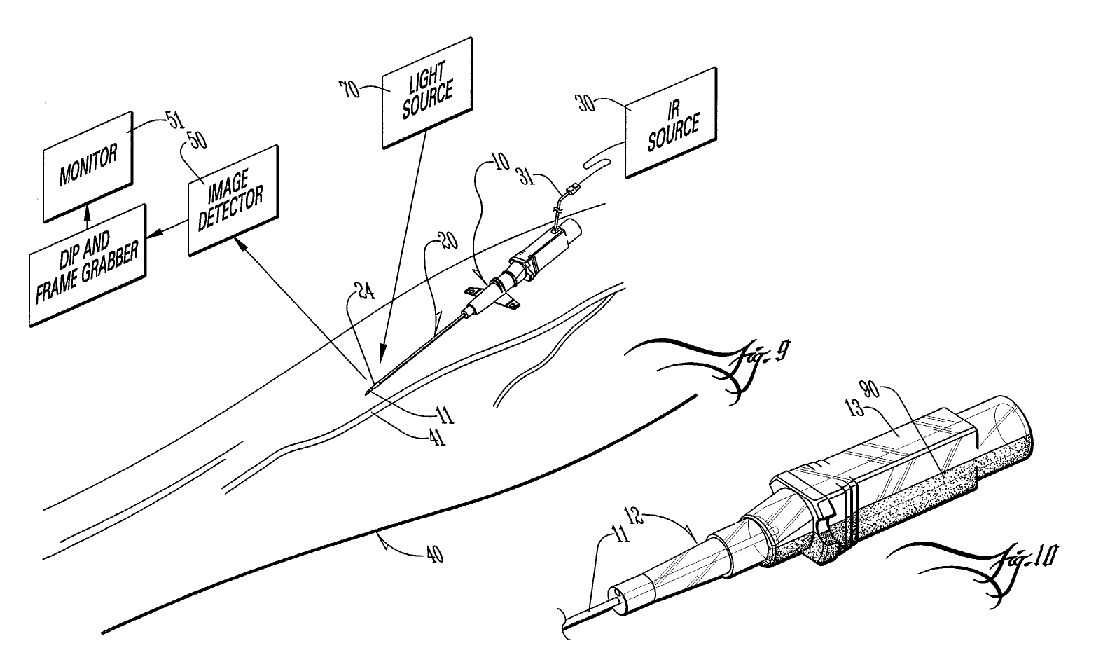

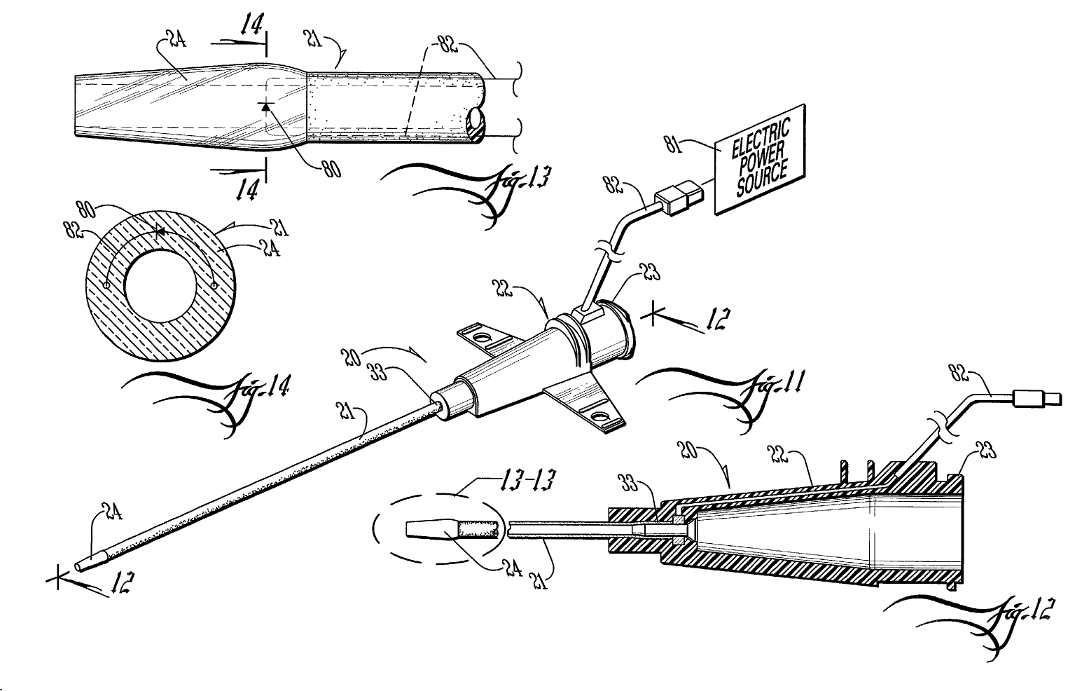

In a further alternative embodiment as shown if FIGS. 11-14, the distal end 24 of the cannula 20 is illuminated by an IR light emitted diode 80 embedded in the distal end 24. An electric power source 81 is operatively coupled through electric wires 82 embedded in the body sheathing portion 22 and the needle sheathing portion 21 of the cannula 20 so as to provide electric power to the light emitting diode 80. Preferably the light emitting diode 80 is a micro-diode. In addition to providing an external power source coupled to the light emitting diode 80 through wired 82 embedded in the cannula 20, power may be provided by an external source coupled electromagnetically to the light emitting diode 80, thus avoiding wires in the cannula 20.

In addition to the embodiment in which a single light emitting diode 80 is employed, other embodiments may include arrays of light emitting diodes arranged in distinctive patterns, such as one or more longitudinally arranged lines. The plurality of light emitting diodes may also be spaced more densely toward the distal end 24 and progressively less densely spaced toward the proximal end of the cannula 20 so that the location of the distal end 24 and the orientation of the cannula 20 may be easily discerned.

An alternative embodiment of the present invention includes a partially opaque flash chamber 13 as illustrated in FIG. 10. Since most flash chambers are transparent, it would be difficult to visualize the blood filling the chamber. In the alternative embodiment of FIG. 10, the flash chamber 13 is provided with a backing 90. The backing 90 is white, or otherwise opaque, to allow the detector to image the blood filling the chamber and verify a successful insertion. The backing 90 may extend along the sides as well as the back of the flash chamber. The backing may be either reflective or absorptive of the radiation being detected so long as it provides a contrast with the optical properties of blood so that the blood is easily distinguishable.

While the preferred embodiments of the invention as described above would use IR radiation, other electromagnetic radiation, including visible light, is contemplated as being within the scope of the present invention.

The present invention has been described with reference to certain preferred and alternative embodiments that are intended to be exemplary only and not limiting to the full scope of the present invention as set forth in the appended claims.

What is claimed is:

1. In an intraluminal indwelling catheter having a body, a hollow needle communicating with the body and a removable cannula enclosing the needle, the improvement comprising:
an electromagnetic radiation signal encoded in the catheter whereby the location of the catheter is detectable by an optical image detector sensitive to said electromagnetic radiation signal.

2. The improvement of claim 1 wherein said electromagnetic radiation signal is an infrared radiation signal.

3. The improvement of claim 2 wherein said infrared radiation signal comprises means for emitting infrared radiation from the distal end of said cannula.

4. The improvement of claim 3 wherein said means for emitting comprises an infrared radiation emitting micro-diode embedded in said distal end of said cannula and means for providing electrical power to said micro-diode.

5. The improvement of claim 3 wherein said means for emitting comprises a source of infrared radiation, means for transmitting said infrared radiation to said catheter and means for coupling said means for transmitting to said cannula for emitting infrared radiation from said distal end of said cannula.

6. The improvement of claim 2 where said infrared radiation signal comprises an array of light emitting diodes on said cannula.

7. The improvement of claim 6 wherein said array of light emitting diodes is spaced more densely toward the distal end of said cannula.

8. The improvement of claim 2 wherein said infrared radiation signal comprises regions of varying optical properties on said cannula forming a pattern that is easily visualized and distinctive from nearby anatomical structures when illuminated by an external source of infrared radiation and viewed on said optical image detector.

9. The improvement of claim 8 wherein said regions of varying optical properties comprises a helical pattern.

10. The improvement of claim 9 wherein said helical pattern comprises one or more solid bands.

11. The improvement of claim 9 wherein said helical pattern comprises a series of helically arranged dots.

12. The improvement of claim 9 wherein said helical pattern comprises a pair of criss-crossing helical bands.

13. The improvement of claim 8 wherein said regions of varying optical properties comprises a zebra stripe pattern.

14. The improvement of claim 2, wherein said catheter further comprises a flash chamber communicating with the body of the catheter, said flash chamber comprising a partially opaque flash chamber having a backing comprising optical properties sufficiently different from the optical properties of blood to allow said optical image detector to image the blood filling the chamber and verify a successful insertion.

15. The combination comprising:
an optical image detector sensitive to electromagnetic radiation, and
an intraluminal indwelling catheter having a body, a needle communicating with the body and a removable cannula enclosing the needle,
wherein said catheter further comprises an electromagnetic radiation signal encoded in the catheter whereby the location of the catheter is detectable by said optical image detector.

16. The combination of claim 15 wherein said electromagnetic radiation signal comprises an infrared radiation signal.

17. The combination of claim 16 wherein said infrared radiation signal comprises means for emitting infrared radiation from the distal end of said cannula.

18. The combination of claim 17 wherein said means for emitting comprises an infrared radiation emitting micro-diode embedded in said distal end of said cannula and means for providing electrical power to said micro-diode.

19. The combination of claim 17 wherein said means for emitting comprises a source of infrared radiation, means for transmitting said infrared radiation to said catheter and means for coupling said means for transmitting to said cannula for emitting infrared radiation from said distal end of said cannula.

20. The improvement of claim 16 where said infrared radiation signal comprises an array of light emitting diodes on said cannula.

21. The improvement of claim 20 wherein said array of light emitting diodes is spaced more densely toward the distal end of said cannula.

22. The combination of claim 16 wherein said infrared signal comprises regions of varying optical properties on said cannula forming a pattern that is easily visualized and distinctive from nearby anatomical structures when illuminated by an external source of infrared radiation and viewed on said optical image detector.

23. The combination of claim 22 wherein said regions of varying optical properties comprises a helical pattern.

24. The combination of claim 23 wherein said helical pattern comprises one or more solid bands.

25. The combination of claim 23 wherein said helical pattern comprises a series of helically arranged dots.

26. The combination of claim 23 wherein said helical pattern comprises a pair of criss-crossing helical bands.

27. The improvement of claim 22 wherein said regions of varying optical properties comprises a zebra stripe pattern.

28. The combination of claim 16 further comprising a partially opaque flash chamber communicating with the body, said partially opaque flash chamber having a backing comprising optical properties sufficiently different from the optical properties of blood to allow said optical image detector to image the blood filling the chamber and verify a successful insertion.
